(19) United States
(12) Patent Application Publication
(10) Pub. No.: US 2020/0281828 A1
(43) Pub. Date: Sep. 10, 2020

(54) MAGNETIC MASCARA COMPOSITIONS AND RELATED METHODS

(71) Applicant: Laura A. Hunter, Mill Creek, WA (US)

(72) Inventor: Laura A. Hunter, Mill Creek, WA (US)

(21) Appl. No.: 16/543,144

(22) Filed: Aug. 16, 2019

Related U.S. Application Data
(60) Provisional application No. 62/814,866, filed on Mar. 7, 2019.

(51) Int. Cl. A61K 8/19; A61K 8/02; A61K 8/26; A61K 8/81; A61Q 1/10

(52) U.S. Cl. CPC A61K 8/19; A61K 8/0241; A41G 5/02; A61K 8/8147; A61Q 1/10; A61K 8/26

(57) ABSTRACT

Various magnetically attachable eyelash prosthetic systems mediated by a magnetic mascara are provided, comprising: (a) a curable, load-bearing magnetic mascara comprising a curable, load-bearing structural adhesive adherable to an eyelash surface that incorporates magnetic particles comprising a ferromagnetic compound; and (b) a magnetic eyelash prosthetic incorporating one or more magnetic elements formed from transition elements, and/or combinations formed from transition elements, post-transition elements and lanthanide elements exhibiting high intrinsic coercivity and positioned at least at the lash base of the eyelash prosthetic, to enable the magnetic attachment of eyelash prosthetics to eyelash-adhered magnetic mascara film. Various methods for producing and using the various magnetically attachable eyelash prosthetics and the various load-bearing magnetic mascaras are also provided.

### CROSS-REFERENCE TO RELATED APPLICATION

This application claims the benefit of U.S. provisional application No. 62/814,866, filed on Mar. 7, 2019, the contents of which are incorporated herein by reference.

### TECHNICAL FIELD

The present disclosure relates to various magnetic compositions for use as curable, load-bearing magnetic mascara formulations for magnetically attaching/detaching eyelash prosthetics for cosmetic enhancement. Methods for making and using the disclosed curable, load-bearing mascara formulations for magnetically and reversibly attaching/detaching one or more eyelash prosthetic units to consumer eyelids and/or native eyelashes are also provided.

### BACKGROUND OF THE INVENTION

The cosmetic industry is estimated to be one of the fastest growing economic sectors, reaching multi-billion dollars in global annual sales. The global demand for innovative beauty products is incessant, and manufacturers are responding to the demands by developing healthier and more visually pleasing products that can provide multiple benefits in comparison to the lackluster performance of yesterday's products.

In particular, beauty formulations that can be applied to the facial skin are especially in demand. Mascara and eyeliners are particularly desirable by consumers because these beauty-enhancing products provide substantial benefits to their consumers by making a visible impact on their appearances, and thereby, improving one's self-confidence. Mascaras can be formulated to coat native eyelashes with highly pigmented and lash-thickening agents to cosmetically exaggerate the thickness and length of native eyelashes with minimal effort. Women population are “demanding” more innovative products capable of taking their ordinary-looking eyelashes to a more favorable presentation of lush/thick/lengthy lashes. Product developers are responding by inventing a broad variety of “cosmetic lashes” or “eyelash prosthetics,” which can be manufactured from human hair, animal hair, insect-derived fiber, and various synthetic blends thereof. Because wearing cosmetic eyelashes can dramatically enhance the size/prominence of the user's eyes, the demand for lash prosthetics is on the rise on a global level, among women of a broad age group. However, conventional eyelash prosthetics pose some inconveniences, which may dissuade a majority of women from trying them at all. For many, the daily application of eyelash prosthetics can be tiresome and time-consuming, requiring a steady pair of hands, and over-exposing the delicate eyelid dermis to potentially toxic glue formulations that may be wrinkle-promoting and difficult to remove.

To avoid these unpleasant user experiences associated with using sticky glue adhesives, alternative lash-enhancing products have been introduced to the market with mixed reviews. Recently, magnetic lashes containing small magnets have been developed that can be superimposed against each other as a top/bottom pair, intended to be positioned over and under native eyelashes for their stabilization. These magnetic lashes are not without technical challenges in applying them, often requiring multiple attempts to properly align the top magnetic lash over the bottom magnetic lash in order to stably secure them around the native eyelashes.

There is an unmet need for advanced mascara compositions capable of stabilizing magnetic eyelash prosthetic products, enhancing the appearance of lash volume (thickness), and facilitating convenient cosmetic enhancement that can be easy to apply and to remove.

### SUMMARY

In several embodiments, the inventive concept is directed to a curable, load-bearing magnetic mascara capable of attaching one or more magnetic eyelash prosthetics to the surfaces of eyelids and native lashes, comprising: (a) one or more magnetic particles suitable for use in a cosmetic formulation; and (b) a curable, load-bearing structural adhesive, capable of adhering to the surface of native eyelashes and capable of magnetically supporting the attachment of a magnetic eyelash prosthetic, wherein the cosmetic formulation is convertible from a non-load-bearing state to a load-bearing adhesive state after coating the native lash surface; and wherein the adhesive force between the load-bearing structural adhesive of the lash-adhered mascara and the native lash surface is greater than the magnetic force between the load-bearing mascara (in a cured state) and the eyelash prosthetic, so that the magnetic eyelash prosthetic(s) can be reversibly attachable and detachable from the surfaces of eyelids and/or native lashes.

In other related embodiments, the inventive concept is directed to a magnetically attachable eyelash prosthetic system comprising two main components: a curable, load-bearing magnetic mascara (“magnetic mascara”) and a magnetically attachable eyelash prosthetic (one or more) comprising a “magnetic lash base,” incorporating magnetic elements. These two magnetic products are designed to magnetically attract each other during intended product use because the magnetic mascara incorporates “magnetic particles” suitable for cosmetics, wherein the “magnetic particles” can magnetically attract the “magnetic lash base” of the eyelash prosthetics, and thereby, supporting the stable attachment of the eyelash prosthetics during consumer “wear”/ product use.

### BRIEF DESCRIPTION OF THE DRAWINGS

FIG. 1 illustrates a side perspective of a hypothetical human face (eye region) before the application of curable, load-bearing magnetic mascara and the magnetic eyelash prosthetic(s) of the present invention.

FIG. 2 illustrates the top-coating of the upper native lashes with a curable, load-bearing magnetic mascara composition as one embodiment.

FIG. 3 illustrates the under-coating of the upper native lashes with a curable, load-bearing magnetic mascara composition as one embodiment.

FIG. 4 illustrates the coating of the upper eyelid with a magnetic or non-magnetic eyeliner as an optional step, as one embodiment.

FIG. 5A illustrates the spatial alignment of a magnetic lash prosthetic over a film (representative of a cured state) formed over native eyelashes by pre-coating with a curable, load-bearing magnetic mascara (without eyeliner application).

FIG. 5B illustrates the spatial alignment of a magnetic lash prosthetic over a film (representative of a cured state) formed over native eyelashes by pre-coating with a curable, load-bearing magnetic mascara and an eyeliner (either magnetic or non-magnetic).

FIG. 6A illustrates the attachment of a magnetic lash prosthetic (FIG. 5A) over the film (representative of a cured state) formed over native eyelashes by pre-coating with a curable, load-bearing magnetic mascara (without eyeliner application).

FIG. 6B illustrates the attachment of a magnetic lash prosthetic (FIG. 5B) over the films (representative of a cured state) formed over native eyelashes by pre-coating with a curable, load-bearing magnetic mascara and an eyeliner (either magnetic or non-magnetic).

FIG. 7 illustrates the attachment of magnetic lash prosthetics from a frontal perspective, showing the lash enhancing effect resulting from the arrangement illustrated in FIG. 6A (left eye, without eyeliner) and FIG. 6B (right eye, with eyeliner).

### DETAILED DESCRIPTION

### A. Definitions

The term “a” refers to one or more of an item/material of interest as typically employed in patent documents.

The term “plurality” refers to one or more of an item/material of interest, interchangeable with the meaning of “many” or “several” or “a set.”

The term “cosmetic” refers to the external application of certain formulations or procedures intended to improve or restore a user's appearance, including the thickening and lengthening appearance of native and prosthetic eyelashes for cosmetic enhancement.

The term “pigment” refers to a coloring agent that can be derived from natural or synthetic sources in order to make the product more desirable from a user's perspective. The pigments suitable for making the curable, load-bearing magnetic mascara can be selected from a range of materials providing the colors of interest, including black, brown, gray, blue, violet, red, green, orange, yellow and various blends thereof.

The term “dermal compatible” refers to the biocompatibility relative to skin tissue for ensuring the safety of product users for the curable, load-bearing magnetic mascara and the magnetic eyelash prosthetics described herein. All ingredients/materials contemplated for the practice of the inventive embodiments are intended to be dermal compatible, and this phrase may be interchangeably used or intended to mean the same relative to the word “cosmetic.”

The terms “cosmetic eyelashes” or “eyelash prosthetics” refer to non-native (manufactured) eyelashes for cosmetic application/adornment/enhancement to improve the user's appearance by thickening and/or lengthening the native eyelashes.

The terms “eyelashes” or “eyelash hairs” are used interchangeably herein to mean “many” or “several” or “a set” of individual hairs derived from human, animals, insects and/or synthetically manufactured blends of natural and man-made materials in any combination or relative ratios.

The term “structural adhesives” refers to an adhesive capable of “hardening” or “curing” into a material capable of holding two or more substrates together in order to bear the forces suitable for the lifetime performance of the product.

The term “curable” refers to the induction of chemical and/or physical reactions and/or processes that can result in the subsequent toughening or hardening of a polymer material by the close association or cross-linking of polymer chains. The term can be applicable for the conversion of a liquid or semi-liquid formulations to a gel-like, semi-solid or solid states.

The term “incorporating” refers to the inclusion or the addition of a material substance of interest in any manner, not limited to any known or unknown processes.

The terms “load-bearing magnetic mascara” or “magnetic mascara” of the present invention refer to mascara formulations incorporating one or more “magnetic particles” exhibiting strong ferromagnetic properties, and capable of supporting the physical attachment of magnetic eyelash prosthetic(s) based on magnetic forces of attraction.

The terms “load-bearing magnetic eyeliner” or “magnetic eyeliner” refer to eyeliner formulation incorporating one or more “magnetic particles” exhibiting strong ferromagnetic properties, and capable of supporting the physical attachment of magnetic eyelash prosthetic based on magnetic forces of attraction. The magnetic eyeliner was claimed/disclosed by the present Inventor/Applicant in a previously filed patent application Ser. No. 16/287,649.

The term “magnetic particles” refers to magnetic materials exhibiting suitable magnetic properties that can be incorporated into the magnetic mascara formulation of the present disclosure, including various ferromagnetic compounds, such as those formed from transition metal oxides, and/or combinations formed from transition metal oxides, post-transition metal oxides and alkaline earth metal oxides, including (without limitation) iron oxides, hematite Fe₂O₃, magnetite Fe^{II}(Fe^{III})₂O₄, super-paramagnetic iron oxides (SPIO), chromium dioxide CrO₂, barium ferrites (BaFe₁₂O₁₉, Ba₂ZnFe₁₉O₂₃, BaFe²⁺₂Fe³⁺₁₆O₂₇), bismuth ferrite (BiFeO₃), manganese-zinc ferrite (MnZn), cobalt ferrite, CoFe₂O₄, strontium ferrite, SrFe₁₂O₁₉, or yttrium iron granate (YIG) Y₃Fe₅O₁₂.

The term “magnetic eyelash prosthetic” refers to the non-native eyelashes formed by incorporating “magnetic elements” into the “magnetic lash base,” which also functions by stably adhering a plurality of non-native eyelashes at their proximal end (opposite end to the eyelash tips). The term “magnetically attachable eyelash prosthetic” can be used interchangeably with “magnetic eyelash prosthetic.”

The term “magnetic lash base” refers to the structural component of the “magnetically attachable eyelash prosthetic” that can stably secure a plurality of eyelash hairs (non-native) at their proximal ends, and can incorporate “magnetic elements” positioned and oriented along the magnetic lash base, exhibiting high intrinsic coercivity in the presence of the “curable, load-bearing magnetic mascara” and/or “curable, load-bearing magnetic eyeliner.” The application of both products is described in FIGS. 4, 5B, and 6B of the present disclosure, and the magnetic eyeliner was claimed/disclosed by the present Inventor/Applicant in a previously filed patent application Ser. No. 16/287,649.

The term “magnetic elements” (incorporated into the “magnetic lash base”) refers to permanent magnets of the hard or soft type, formed from transition elements, and/or combinations formed from transition elements, post-transition elements and lanthanide elements, including without limitation iron, cobalt, nickel; alloys, such as samarium-cobalt alloys, including SmCo₅, Sm₂Co₁₇, optionally alloyed with Fe, Cu, and Zr; and compounds such as neodymium-iron-boron Nd₂Fe₁₄B optionally alloyed with Pr and Dy, commonly referenced as “rare-earth” or “super magnets”; and/or other alloys such as bismanol and AlNiCo.

The term “magnetic” and “ferromagnetic” are used interchangeably in this disclosure without any limitations.

The term “ferromagnetic compound” is used to distinguish from “ferromagnetic elements” and “ferromagnetic alloys” in that compounds can be composed of two or more chemical elements and held together by chemical bonds. In contrast, “ferromagnetic elements,” such as iron, nickel, and/or cobalt can each be formed from a single element. Furthermore, an “alloy” can be a solid solution of two or more metals with a metallic bonding character. By definition, all iron oxides are deemed to be “ferromagnetic compounds,” whereas iron powders and iron filings obtained by physically grinding elemental iron are deemed to be “ferromagnetic elements.” Finely ground iron is known to be very susceptible to oxidation when exposed to ambient air and water, which can cause discoloration, reduce shelf-life, and potentially impair their intended performance of a given product, and therefore, iron powders/filings are not suitable for making the magnetic mascaras of the present invention.

The terms “low,” “intermediate,” and “high” intrinsic coercivities are used to categorize soft, semi-hard, and hard magnetic materials, wherein “soft” magnetic materials can exhibit a “low” coercivity range of about<1000 Nm, “semi-hard” magnetic magnetic materials can exhibit an “intermediate” coercivity range of about 1000 Nm-10000 A/m, and “hard” magnetic materials can exhibit a “high” intrinsic coercivity range exceeding about>10000 Nm.

The terms “attachable” or “adherable” are used interchangeably in reference to the ability to physically interact with another surface of interest resulting in the bonding of two contacting surfaces, wherein the bonding is reversible as used in relation to the magnetic interaction between the “load-bearing magnetic mascara” coated over native eyelashes (in the cured state) and the “magnetic eyelash prosthetic.”

The term “non-load-bearing” state refers to a state in which a load cannot be supported by an adhesive, as referenced herein.

### B. The Magnetically Attachable Eyelash Prosthetic System Comprising a Magnetic Mascara

Consumers of eyelash-enhancing cosmetic products are very interested in using more advanced products having desirable properties that can be more suitable for their busy schedules and adaptations to various environmental circumstances. Women consumers especially appreciate beauty/cosmetic products that are intelligently designed, lighter in weight, healthier, aesthetically pleasing, comfortable and convenient to use (i.e., “user friendly” in meeting customer expectations). In particular, most women enjoy enhancing their appearance if the process/treatment is not challenging to implement. Many women choose not to fuss with cosmetic lashes and sticky glues because the application process is not very convenient, especially in waiting for glues to dry and requiring manual dexterity, which may be wanting. The present disclosure provides a convenient way for adhering eyelash prosthetics to consumers' eyelids and native eyelashes to significantly enhance the appearance of voluminous lashes without the mess or time wasted in fiddling with tacky/noxious glues, which can be either unhealthy or inconvenient to consumers.

The inventive concept is directed to a magnetically attachable eyelash prosthetic system comprising two main components: a curable, load-bearing magnetic mascara (“magnetic mascara”) and a magnetically attachable eyelash prosthetic (one or more) comprising a “magnetic lash base.” These two magnetic products are designed to magnetically attract each other during intended product use because the magnetic mascara incorporates “magnetic particles” suitable for cosmetics, wherein the “magnetic particles” can magnetically attract the “magnetic lash base” of the eyelash prosthetic, and thereby, supporting the stable attachment of the eyelash prosthetic(s).

In several embodiments, the present invention is directed to curable, load-bearing magnetic mascaras capable of attaching one or more magnetic eyelash prosthetics, comprising: (a) one or more magnetic particles suitable for use in a cosmetic formulation; and (b) a curable, load-bearing structural adhesive, capable of adhering to the surface of native lashes and capable of magnetically supporting the attachment of a magnetic eyelash prosthetic, wherein the cosmetic formulation is convertible from a non-load-bearing state (i.e., non-dry, liquid or semi-liquid) to a load-bearing adhesive state (i.e. cured, dry, hardened, gel-like, semi-solid, or solid) after coating the native lash surface; and wherein the adhesive force between the load-bearing structural adhesive of the lash-adhered mascara and the eyelash hair surface is greater than the magnetic force between the load-bearing mascara (pre-coated, cured state) and the eyelash prosthetic, so that the eyelash prosthetic is reversibly attachable and detachable to/from the consumer's native eyelashes.

In several embodiments, the present invention is directed to a magnetically attachable eyelash prosthetic system, comprising: (a) a lash base incorporating one or more magnetic elements exhibiting high intrinsic coercivity; and (b) a plurality of eyelash hairs adhered at the proximal end to the lash base, wherein the magnetic elements are positioned in a portion of the lash base, and oriented to juxtapose against the surface of lashes pre-coated with a magnetic mascara.

In several embodiments, various methods for producing and using the various magnetically attachable eyelash prosthetics and the various curable, load-bearing magnetic mascara are provided throughout the specification. Furthermore, examples 1-5 provides exemplary formulations for forming curable, load-bearing magnetic mascaras of the present disclosure.

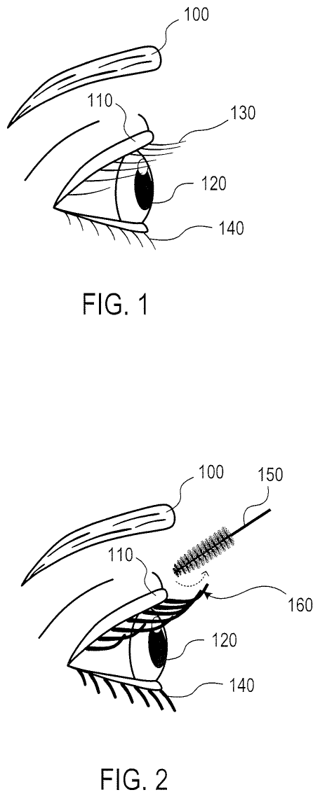

The magnetic interaction between the main components of the magnetically attachable eyelash prosthetic system comprising a magnetic mascara can be explained using illustrations of FIGS. 1-7, described below.

FIG. 1 illustrates a side perspective of a hypothetical human face (eye region) before the application of curable, load-bearing magnetic mascara and the magnetic eyelash prosthetics of the present invention. In FIG. 1, a human eye 120 comprising an upper eyelid 110 that supports the growth of upper native lashes 130 and a row of lower eyelashes 140 is shown, directly below the eyebrow 100. Prior to application, the lashes 130 can be cleaned and/or pre-treated (or both) to remove residual oil and/or previously applied cosmetics, so that the magnetic mascara can optimally adhere to the pre-cleaned and/or pre-treated lash surface.

FIG. 2 illustrates the top-coating of the upper native lashes with a curable, load-bearing magnetic mascara composition as one embodiment. In FIG. 2, the upper lashes 130 (shown in FIG. 1) can be coated over the top surface 160 with a curable, load-bearing magnetic mascara composition 150 comprising ferromagnetic particles, which can be selected from suitable ferromagnetic compounds, such as those formed from transition metal oxides, and/or combinations formed from transition metal oxides, post-transition metal oxides and alkaline earth metal oxides, including without limitation iron oxides, such as hematite Fe₂O₃, magnetite Fe^{II}(Fe^{III})₂O₄, super-paramagnetic iron oxides (SPIO), chromium dioxide CrO₂, barium ferrites (BaFe₁₂O₁₉, Ba₂ZnFe₁₈O₂₃, BaFe²⁺₂, Fe³⁺₁₆O₂₇), bismuth ferrite (BiFeO₃), manganese-zinc ferrite (MnZn), cobalt ferrite, CoFe₂O₄, strontium ferrite, SrFe₁₂O₁₉, or yttrium iron granate (YIG) Y₃Fe₅O₁₂. The transition metal oxides, and/or combinations of transition metal oxides, post-transition metal oxides and alkaline earth metal oxides can be of at least binary and/or ternary nature. The magnetic mascara-coated lashes appear more pigmented and thicker, as shown. The top-coated “magnetically loaded” lashes can magnetically attach the magnetic eyelash prosthetics comprising magnetic elements described herein.

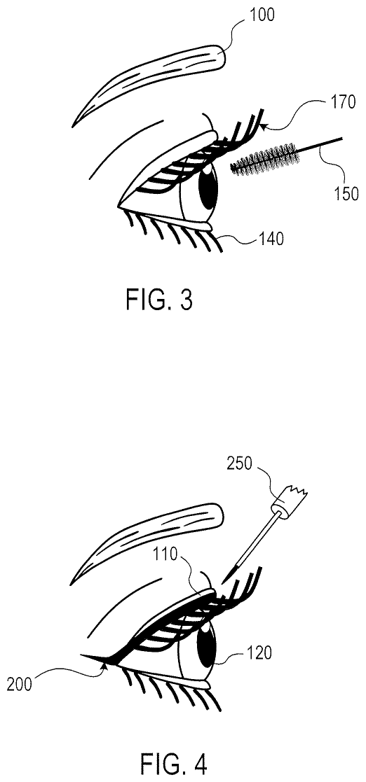

FIG. 3 illustrates the under-coating of the upper native lashes with a curable, load-bearing magnetic mascara composition as one embodiment. In FIG. 3, the upper lashes 130 (as shown in FIG. 1) can be coated over the under surface 170 of the lash hairs with a magnetic mascara composition 150 comprising ferromagnetic particles as described in FIG. 2, resulting in more pigmented and thicker/longer magnetic mascara-coated native lashes that can be visually prominent in comparison to the situation without cosmetic adornment (as compared to uncoated lashes in FIG. 1).

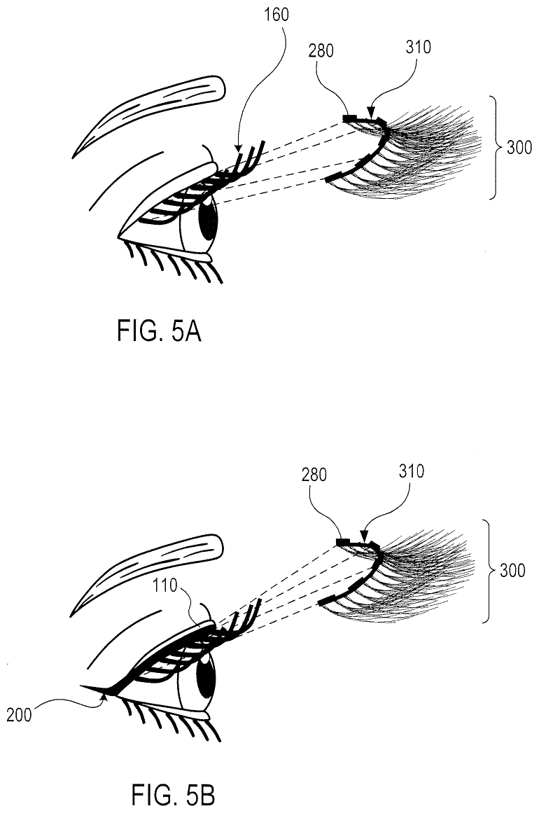

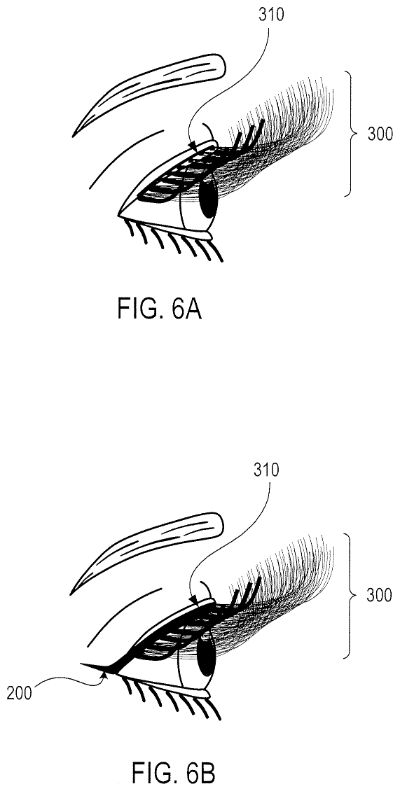

FIG. 4 illustrates the coating of the upper eyelid with a magnetic or non-magnetic eyeliner as an optional step, as one embodiment. In FIG. 4, the upper eyelid 110 (shown in FIG. 1) can be coated with a magnetic eyeliner 250 (applicator brush shown) to form an eyeliner film 200 that can be shaped to a design of interest. The magnetic eyeliner comprises ferromagnetic particles selected from suitable magnetic compounds, such as those formed from transition metal oxides, and/or combinations formed from transition metal oxides, post-transition metal oxides and alkaline earth metal oxides, including without limitation iron oxides, such as hematite Fe₂O₃, magnetite Fe^{II}(Fe^{III})₂O₄, super-paramagnetic iron oxides (SPIO), chromium dioxide CrO₂,barium ferrites (BaFe₁₂O₁₉, Ba₂ZnFe₁₈O₂₃, BaFe²⁺₂Fe³⁺₁₆O₂₇), bismuth ferrite (BiFeO₃), manganese-zinc ferrite (MnZn), cobalt ferrite, CoFe₂O₄, strontium ferrite, SrFe₁₂O₁₉, or yttrium iron granate (YIG) Y₃Fe₅O₁₂. The transition metal oxides and/or combinations of transition metal oxides, post-transition metal oxides and alkaline earth metal oxides can be of at least binary and/or ternary nature. The pre-formed magnetic eyeliner 200 can magnetically interact with the magnetic elements incorporated within the lash base of the magnetic eyelash prosthetics (as further described in FIGS. 5B and 6B). Alternatively, a non-magnetic eyeliner may be applied for aesthetic purposes, or a combination of magnetic and non-magnetic eyeliners. The application of the eyeliner can be performed optionally before the application of the magnetic mascara of the present invention (as shown in FIGS. 2-3) or subsequently to mascara application.

FIG. 5A illustrates the spatial alignment of a magnetic lash prosthetic over a film formed over native eyelashes by pre-coating with a curable, load-bearing magnetic mascara (without eyeliner application). In FIG. 5A, the magnetic lash prosthetic 300 comprising a magnetic lash base 310 that can interact with the top surface of native lashes 160 as shown. The eyelash prosthetic 300 comprises a magnetic lash base 310 that can stably secure a set of eyelash hairs as shown. The magnetic lash base 310 can incorporate one or more magnetic elements 280, comprising variably sized microscopic (powderized/ micronized) and/or macroscopic (sub-millimeter and/or millimeter sized) ferromagnetic elements, and ferromagnetic alloys or ferromagnetic compounds that can exhibit strong magnetic properties. The magnetic elements 280 can be structurally arranged in various ways, to facilitate different spatial magnetic field distributions that can enable more directed forms of magnetic attachment as desired by consumers.

FIG. 5B illustrates the spatial alignment of a magnetic lash prosthetic over a film formed over native eyelashes by pre-coating with a curable, load-bearing magnetic mascara and an eyeliner (either magnetic or non-magnetic). In FIG. 5B, the magnetic lash prosthetic 300 comprising a magnetic lash base 310 that can interact with the top surface of native lashes 160 as shown. The eyelash prosthetic 300 comprises a magnetic lash base 310 that can stably secure a set of eyelash hairs as shown. The magnetic lash base 310 can incorporate one or more magnetic elements 280, comprising variably sized microscopic (powderized/micronized) and/or macroscopic (sub-millimeter and/or millimeter sized) ferromagnetic elements and ferromagnetic alloys or ferromagnetic compounds that can exhibit strong magnetic properties. The magnetic elements 280 can be structurally arranged in various ways, to facilitate different spatial magnetic field distributions that can enable more directed forms of magnetic attachment as desired by the consumers.

FIG. 6A illustrates the attachment of a magnetic lash prosthetic (FIG. 5A) over the film formed over native eyelashes by pre-coating with a curable, load-bearing magnetic mascara (without eyeliner application). In this embodiment, the magnetic mascara enables the attachment of the prosthetic lashes independent of a magnetic eyeliner. Furthermore, the magnetic mascara can be formulated to include a variety of natural and synthetic fibers, or their respective blends, that can be coated over native lashes to enhance the volume/thickness/length of the native lashes.

FIG. 6B illustrates the attachment of a magnetic lash prosthetic (FIG. 5B) over the films formed over native eyelashes by pre-coating with a curable, load-bearing magnetic mascara and an eyeliner (either magnetic or non-magnetic). In this embodiment, the application of the magnetic mascara further augments the stabilization/attachment of the prosthetic lashes in conjunction with the application of a magnetic eyeliner. The combined application of a magnetic mascara and magnetic eyeliner to the native lashes and eyelid skin can be expected to expand the surface area available for attaching a magnetic eyelash base, which can effectively strengthen the degree of attachment and can minimize inadvertent repositioning of the magnetic lash base during consumer use. In addition, the combined application of a magnetic mascara and magnetic eyeliner may support the co-attachment of one or several prosthetics, or differently styled lash prosthetics, or additional magnetic adornments to the pre-coated eyelid skin and native lashes. Furthermore, the magnetic mascara can be formulated to include a variety of natural and synthetic fibers, or their respective blends, that can be coated over native lashes to enhance the volume/thickness/length of the native lashes. A broad range of pigment combinations are contemplated to provide different aesthetic effects, including the enhancement in color, luster, and iridescence. As one embodiment, magnetic particles can be provided as a pigment in powdered form, and directly applied to the pre-coated (magnetic) eyelid skin and/or native lashes such as to adhere by magnetic forces. The aforementioned magnetic particles can also be provided pre-coated, so as to alter the native color, luster, and iridescence thereof.

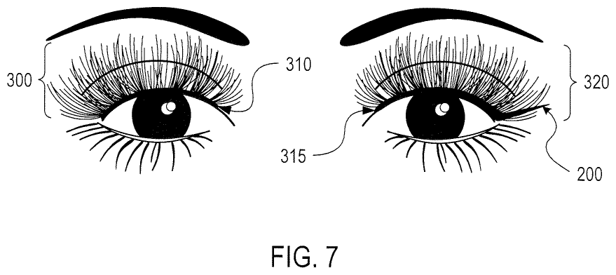

FIG. 7 illustrates the attachment of magnetic lash prosthetics from a frontal perspective, showing the lash enhancement effect resulting from the arrangement illustrated in FIG. 6A (left eye, without eyeliner) and FIG. 6B (right eye, with eyeliner). In this embodiment, the dramatic effect of magnetically attaching lash prosthetics can be appreciated by utilizing the magnetic mascara without eyeliner application (left eye) and by applying both magnetic mascara and eyeliner (right eye).

### DETAILED DESCRIPTION

### C. Functional Components of a Curable, Load-Bearing Magnetic Mascara Formulations

### Exemplary Structural Adhesives

In general, adhesives can be defined as substances capable of holding at least two surfaces together. A wide range of adhesive formulations are conceivable, and can be differentiated into either “pressure-sensitive adhesives” (PSAs) and “structural adhesives.” Pressure-sensitive adhesives (PSAs) can adhere strongly to solid surfaces upon application of light contact pressure and short contact duration, useful for manufacturing “post it” notes, adhesive pads, pressure-sensitive tapes, various labels, and a wide variety of products. Three different types of PSAs commercially employed are described as either: a) organic solvent based, b) water-based (emulsion), or c) thermoplastic. Pressure-sensitive adhesives are appreciated for their ease of application and removal from surfaces, but exhibit relatively less load-bearing capability than structural adhesives, and can be more prone to inadvertent detachment from substantially moist/oily surfaces, similar to most skin or hair surfaces. Alternatively, structural adhesives can be defined as adhesives that can “harden” or “cure” into a material capable of stably holding two or more substrates together during the expected lifetime of the product. Structural adhesives are often termed “load-bearing” adhesives, and exhibit higher load-bearing capabilities than aforementioned PSAs. Structural adhesives can be formulated utilizing various polymer chemistries, including polyurethane-, acrylic-, silicone-, vinyl-, or epoxy-based polymers and blends, among other suitable materials known by persons skilled in the art.

With respect to skin-contacting and hair-contacting adhesive formulations, a variety of different types of adhesives can be utilized to enable skin/hair-bonding. Suitable examples include various temporary dermal adhesives intended to support wound closure/wound repair. These formulations utilize among others, cyanoacrylate monomers and monomer blends formed from 2-octyl cyanoacrylate (CAS 133978-15-1) and/or n-Butylcyanoacrylate. Other dermal formulations may include Octyl 2-cyanoacrylate (CAS 6701-17-3), Isobutyl 2-cyanoacrylate and various materials that are functional equivalents.

In selecting suitable skin-contacting and hair-contacting adhesives, cyanoacrylate formulations may be limited for use as a dermal adhesive in that they can be polymerized in the presence of water, and can polymerize rapidly (from tens of seconds to few minutes) in a manner that can be challenging to control for skin/hair applications. These formulations must be stored dry to prevent their activation, since they can initiate curing upon contact with a moist skin/hair. Thus, they must be applied comparatively quickly, and spread accurately. Because their adhesion to skin/hair is quite substantial, and can last for a comparatively long time period (approximately one to several days), such formulations can be predictably suitable for formulating dermal adhesive products, in that: a) these formulations will not permit a sufficiently intermittent cosmetic application during 4-12 hour period, and b) the premature removal from a skin surface may require substantial force to be exerted and result in substantial discomfort. Cured formulations are also difficult to remove by traditional detergents, including soap and water, so that more volatile and potentially hazardous solvents, such as ethanol, ethyl acetate, or acetone may be needed to remove films/layers formed on the skin/hair from such formulations. However, these types of solvents are known to degrease the skin/hair and may cause skin/hair irritations. Because the skin/hair of some consumers can be very sensitive, it is desirable to avoid using such volatile, irritating and potentially hazardous solvents during dermal adhesive product use/product removal. Thus, there is a specific need to find more suitable skin-adhering and hair-adhering formulations that do not detach as easily from the skin/hair as aforementioned PSAs, and that do not adhere as strongly, as in the example of the aforementioned cyanoacrylate-based structural adhesives.

### Exemplary Pigments

The addition of pigments enables the mascara product to be formed in any desirable color from a spectrum of hues. Suitable pigments can be chosen from natural or synthetic, metallic, nonmetallic, inorganic pigments, organic pigments, magnetic, nonmagnetic and/or pearlescent pigments, depending on the desired visual appearance, flowability, spreadability, dispersability, and/or wetting ability. Suitable pigments can be provided a) in various particle size ranges from typically tens of nanometers to about several hundred micrometers; b) with or without surface treatments; c) in hydrated or dehydrated form; and d) as is, or blended with each other, to reach a desired color spectrum or other property. Exemplary inorganic pigments include black, yellow, red and brown iron oxides; titanium dioxide (rutile or anatase) (white), manganese violet; ultramarine blue; chromium oxide (green) and ferric blue, among others. Organic pigments include pigments D & C yellow, D & C orange, D & C red, carbon black and other dyes and/or lakes, among others. Pearlescent pigments include white pearlescent pigments, such as mica coated with titanium oxide or bismuth oxychloride; colored pearlescent pigments such as titanium mica with iron oxides, titanium mica with ferric blue or chromium oxide, titanium mica with an organic pigment of the abovementioned type, and pigments based on bismuth oxychloride, among others. Suitable pigment particles can exhibit an average diameter size selected from several options, including for example, ranging from about 1 μm to 1 mm, from about 1 μm to 500 μm, from about 500 μm to 1 mm, and from about 1 μm to 100 μm. As another embodiment, the pigment particles exhibit an average diameter size ranging from about 100 nm to 1 μm, wherein the pigment particles can be further encapsulated to inhibit sub-dermal transport.

### Exemplary Magnetic Particles and Magnetic Elements

Magnetic materials differ widely in their capacity to be magnetized or demagnetized. Materials that can be more susceptible for magnetization and demagnetization are commonly referred to as magnetically “soft” materials, whereas magnetic materials that are comparatively “harder” to magnetize and demagnetize are commonly referred to as magnetically “hard” materials.

A measure of the ability of a magnetic material to withstand an external magnetic field without becoming demagnetized is referenced as the “intrinsic” coercivity (Hci). The intrinsic coercivity (Hci) not only allows the magnet to withstand a demagnetizing field, but also provides a measure of stability under other adverse circumstances such as being less susceptible to demagnetization at elevated temperatures, demagnetization due to vibration, and demagnetization due to radiation exposure.

Magnetically hard materials can be used to form permanent magnets. The purpose of a permanent magnet is to provide a magnetic field in a particular volume of space. Permanent magnets require a “high” intrinsic coercivity, because a permanent magnet, once magnetized, is intended to resist the demagnetizing effect of ambient fields, including its own. An “infinite” intrinsic coercivity (Hci) would mean that the magnet is truly “permanent,” meaning that the relative measure of the capability of magnets to uphold its own state of magnetization is directly correlated with their intrinsic coercivity values.

The differentiation between materials of low and high coercivity in the context of this application can be understood as (a) the degree of softness versus hardness of a magnetic material, and (b) the degree of permanence, expressed as the intrinsic coercivity of the magnetic field generated by a given magnet. The above considerations are based on the reference “Introduction to Magnetic Materials,” (B. D. Cullity and C. D. Graham, 2nd Ed., IEEE Press and John Wiley & Sons, Princeton, N.J.; 2009).

Ferromagnetic alloys can exhibit hard or soft magnetism depending on their coercivity. “Hard magnetic alloys (also called magnetically hard alloys) have sufficiently high coercive force as a resistance to demagnetizing fields with coercivities exceeding 10 kA m⁻¹” as referenced in “Bulk nanocrystalline and nanocomposite alloys produced from amorphous phase” (A. Inoue, D. V. Louzguine, Nanostructured Metals and Alloys, 2011). Thus, magnetic materials having coercivity values above about 10⁴ A/m are deemed to be “hard,” the “soft” magnetic materials have a coercivity less than about 10³ A/m, and the interim region ranging from about 10³ A/m to about 10⁴ A/m can be often referred to as “semi-hard.”

Within the scope of the underlying application, the terms low, intermediate and high intrinsic coercivity are meant to relate to the categories of soft-, semi-hard-, and hard magnetic materials, exhibiting a “low” coercivity of about<1000 A/m corresponding with soft magnetic materials, an “intermediate” coercivity in the range of about 1000 A/m-10000 A/m corresponding with semi-hard magnetic materials, and a “high” coercivity exceeding about>10000 A/m corresponding with hard magnetic materials, respectively.

For producing the magnetically attachable eyelash prosthetic system, it is desirable to select (a) suitable “magnetic particles” exhibiting strong ferromagnetic properties for incorporation within the magnetic mascara formulations; and (b) suitable “magnetic elements” exhibiting strong ferromagnetic properties for incorporation within the “magnetic lash base.” The “magnetic particles” in the magnetic mascara formulation and the “magnetic elements” in the eyelash base can be employed as: (i) a filler agent, as (ii) a color-imparting component (or pigment), and (iii) a magnetic material that can facilitate the anchoring/attachment of the “magnetic lash base” to the present “curable, load-bearing magnetic mascara.”

The ferromagnetic properties of the “magnetic particles” and the “magnetic elements” must be compatible in order for the magnetic eyelash prosthetic to attach stably to the magnetic mascara (lash-adhered/pre-coated). As an embodiment, a ferromagnetic material exhibiting an intrinsically high coercivity is suitable for use in forming “magnetic particles” incorporated within the “curable, load-bearing magnetic mascara” formulation, for example, selected from the range from about 10-15,000 kA/m, from about 10-10,000 kA/m, and from about 10-5,000 kA/m. As an embodiment, a “magnetic element,” having an intrinsically high coercivity is incorporated into the “magnetic lash base,” selected for example, ranging from about 10-15,000 kA/m, from about 10-10,000 kA/m, and from about 10-5,000 kA/m. The ferromagnetic materials can be selected from ferromagnetic elements, ferromagnetic alloys and ferromagnetic compounds, and are preferably selected to withstand particular environmental and storage conditions during the expected time frame of their use and shelf-life period.

As another embodiment, suitable materials for forming the “magnetic elements” for incorporation into the “magnetic lash base” include variably sized microscopic (powderized/micronized) and/or macroscopic (sub-millimeter and/or millimeter sized) ferromagnetic elements, ferromagnetic alloys and ferromagnetic compounds, typically used in permanent magnets of the hard or soft type, formed from transition elements, and/or combinations formed from transition elements, post-transition elements and lanthanide elements, including without limitation, iron, cobalt, nickel; alloys, such as samarium-cobalt alloys, including SmCo₅, Sm₂Co₁₇, optionally alloyed with Fe, Cu, and Zr; and compounds such as neodymium-iron-boron Nd₂Fe₁₄B optionally alloyed with Pr and Dy, commonly referenced as “rare-earth” or “super magnets”; and/or other alloys such as bismanol and AlNiCo. Alternatively, the “magnetic elements” can be formed from a combination of at least one or more transition elements, post-transition elements and lanthanide elements. In addition, the combination of transition elements, post-transition elements and lanthanide elements can be of at least binary and/or ternary nature.

As another embodiment, suitable materials for forming “magnetic particles” for incorporation into the “magnetic mascara” include ferromagnetic compounds, such as those formed from transition metal oxides, and/or combinations formed from transition metal oxides, post-transition metal oxides and alkaline earth metal oxides, including without limitation oxide-based magnetic pigments, such as iron oxides, hematite Fe₂O₃, magnetite Fe^{II}(Fe^{III})₂O₄, super-paramagnetic iron oxides (SPIO), chromium dioxide CrO₂, barium ferrites (BaFe₁₂O₁₉, Ba₂ZnFe₁₈O₂₃, BaFe²⁺₂Fe³⁺₁₆O₂₇), bismuth ferrite (BiFeO₃), manganese-zinc ferrite (MnZn), cobalt ferrite, CoFe₂O₄, strontium ferrite, SrFe₁₂O₁₉, or yttrium iron granate (YIG) Y₃Fe₅O₁₂. Alternatively, the “magnetic particles” can be formed from a combination of at least one or more transition metal oxides, post-transition metal oxides and alkaline earth metal oxides. In addition, the combination of at least one or more transition metal oxides, post-transition metal oxides and alkaline earth metal oxides can be of at least binary and/or ternary nature.

As another embodiment, the “magnetic elements” and “magnetic particles” can be provided in encapsulated or unencapsulated form to optimize properties such as suspendability, dispersability, surface wetting characteristics, dermal compatibility, and resistance from environmental factors, including oxidation resistance. For example, iron oxides can be prevented from environmental degradation or direct skin contact by the addition of masking agents, including fatty acids, such as undecylenic, lauric or oleylic acid. Ferromagnetic iron oxide-based compounds are contemplated to be particularly preferable over elemental forms of ferromagnetic materials, such as iron powders, due to their increased oxidation and corrosion resistance, as well as superior shelf life stability and their ability to be effectively dispersed and stabilized by aforementioned masking agents.

With regard to suitable “magnetic particle” sizes for use in the magnetic mascara formulation, generally, the preferred size ranges cannot easily be visually distinguished from one another by the naked eye, thus avoiding a granular appearance. As several embodiments, suitable magnetic particles can exhibit an average diameter size selected from several options, including for example, ranging from about 1 μm to 1 mm, from about 1 μm to 500 μm, from about 500 μm to 1 mm, and from about 1 μm to 100 μm. As another embodiment, the magnetic particles exhibit an average diameter size ranging from about 100 nm to 1 μm, wherein the magnetic particles can be further encapsulated to inhibit sub-dermal transport.

Black iron oxides, which are available in various size ranges and chemistries, are deemed particularly useful, because they impart not only a rich black color, but also confer the desired magnetic properties to the underlying formulation. A suitable overall composition range for such iron oxide based magnetic particles can range from between 10-60 (w/w %), more preferably 20-50 (w/w %), and most preferably from 25-45 (w/w %). It should be noted, that the higher the coercivity of the “magnetic particle” chosen as the pigment component, the lesser amount need be incorporated to achieve a desired magnetic adhesion force for stably attaching a “magnetic lash base” to a complementarily formulated “magnetic mascara.” An analogous rationale can be applied with respect to the incorporation of magnetic elements into the “magnetic lash base.” Thus, the needed magnetic adherent force can be finely controlled through material selection and the relative amount of “magnetic elements” added to the “magnetic lash base” with respect to the amount of “magnetic particles” added to the “magnetic mascara.”

### Suitable Adhesive and Viscosifying Components

The inventor of the present application has found that suitable skin-adhering and hair-adhering formulations can be formed on the basis of acrylate polymer blends, preferably from methyl and ethyl acrylate, 2-hydroxyethylacrylate, butyl acrylate, isobutyl acrylate, t-butyl methacrylate, isooctyl acrylate, ethyl hexyl acrylate, isobornyl acrylate, vinyl acetate, and more particular from: ethyl hexyl acrylate copolymers in an overall weight ratio of 0-30 (w/w %), more preferably 5-25 (w/w %) and most preferably from 10-20 (w/w %); in combination with butyl acrylate, methyl methacrylate, and methacrylic acid copolymers; and methacrylic acid homopolymers in an overall weight ratio of 0-10 (w/w %), more preferably 0-5 (w/w %) and most preferably from 0.5-2.5 (w/w %), that can be selected from a molecular weight range of 5,000-30,000 g/mol, more preferably from 10,000-20,000 g/mol and most preferably from 15,000-20,000 g/mol. The latter polymer blends can be particularly suitable for forming a curable, structural adhesive capable of stably adhering to native eyelashes.

### Suitable Thickening Agents

Depending on the desired flowability and spreadability for an optimized application to lash hairs, viscosifying-, gelling-, thickening-, bulking- and/or thixotropic agents can be added to facilitate the blending with other acylates in the liquid formulations. Examples include gelling agents based on natural gums, including carob gum, guar gum, gum arabic, karaya gum, gum tragacanth and ghatti gum, agar-agar, carrageenans, alginates, gelatin, caseinates, albumins, pectins, starches, polysaccharides, such as xanthan gum, chitin and/or chitosanes, polydextranes such as carboxymethyl dextranes, cellulose and cellulose derivatives, such as carboxymethylcellulose, hydroxypropylcellulose, methylcellulose, hydroxyl-propylmethylcellulose or hydroxyethylcellulose, and cellulose derivatives modified by alkyl- or alkoxy-groups. Others include inorganic filler materials, and/or pigments, such as silicate minerals, talcs or hydrated magnesium silicates, micas or aluminosilicates, such as for example muscovite, margarite, roscoelite, lipidolite, biotite, sericite, hectorite and/or bentonite, kaolin or hydrated aluminium silicate, boron nitride, fumed silica and/or titanium dioxide.

### Suitable Film-Forming Components

In formulating the curable, load-bearing magnetic mascara, other desirable properties include cosmetic functions such as film-forming and opacifying-controlling properties that can facilitate the formation of a homogenous, visually pleasing opaque film surface. The inventor of the present application noted that these properties can be enhanced through the addition of styrene/methacrylate/acrylate copolymer blends, including, but not limited to methacrylic acid-styrene copolymer and/or their respective alkali salts. Suitable overall weight ratios in a formulation comprising such styrene/acrylate copolymer blends can include 5-45 (w/w %), more preferably 10-30 (w/w %) and most preferably from 15-25 (w/w %). In the event, that more water resistant film-forming properties are desirable, fluorinated acrylate polymers, such as 2,2,2-Trifluoroethyl methacrylate, 2,2,3,3-Tetrafluoropropyl methacrylate, 2,2,3,4,4,4-Hexafluorobutyl methacrylate, 2,2,3,3,3-Pentafluoropropyl acrylate, and/or 1,1,1,3,3,3-Hexafluoroisopropyl acrylate can be added to the blend in a suitable proportion to weatherproof the surface. Optionally, to prevent the potential adherence of eyelash hairs to one another during the drying/curing/film forming process, and/or to ease the subsequent removal of the dried/cured mascara from the eyelash hairs after initial application, the amount of film-forming polymers in the mascara can be further reduced or even removed entirely from the formulation.

### Suitable Water Content

Overall water weight ratios for the provided formulations can be chosen from between 0-50 (w/w %) more preferably from 10-40 (w/w %) and most preferably from 25-35 (w/w %). Upon application, the water evaporates up to a residual content (e.g., equivalent to the moisture retained in the skin), substantially solidifying the adhesive formulation in the process, and thereby creating a stable structural support and adherence on lash hair substrates.

### Desired Viscosity Ranges

The viscosity can be adjusted through a desired amount of viscosifying agents, thickening agents, and/or filling agents, particularly including various pigments. The viscosity of the formulations in the underlying disclosure typically can range from 5 cps-300 cps, which can be measured between about the viscosity of water and castor oil (or higher viscosity).

The aforementioned polymer blends do not cure in the presence of water because they are not provided as a monomer, and are already cured. They can be readily dissolved in water, forming viscous to gel-like solutions and are suitably provided in a chemically non-crosslinked state. Because the formulation is formed partially from water-soluble, chemically non-crosslinked polymers, the magnetic mascara film/layer can be easily removed from the skin by employing conventional detergent solutions and associated products intended for eye make-up removal. Because the adhesive formulations in the underlying disclosure harden or cure by means of physical association/crosslinking, effected through solvent evaporation, adhesive and cohesive strength can be substantially improved over semi-liquid and/or gel-like adhesive formulations that do not cure, and thus maintain their relative state throughout the application.

### Emollients/Humectants

To further control the residual moisture content and hardness of the adherent layer, various humectifying-, emollifying-, softening- and/or anti-caking agents can be added to the mascara and eyeliner formulations. Suitable humectants can include ethylene-, propylene-, butylene- and hexylene glycols, as well as their multifunctional alcohol derivatives, alpha-hydroxy acids such as lactic acid, glyceryl triacetate, polymeric polyols such as polydextrose, sodium hexametaphosphate, sugar alcohols such as glycerol, sorbitol, xylitol, maltitol, and urea. Suitable emollients can include castor oil, cetyl alcohol, cetearyl alcohol, cocoa butter, isopropyl myristate, isopropyl palmitate, lanolin, liquid paraffin, polyethylene glycols, shea butter, silicone oils, stearic acid, and stearyl alcohol, as well as C14-18 and C16-18 fatty alcohols and mixtures thereof. Suitable humectifying-, emollifying-, softening- and/or anti-caking agents concentrations in the overall composition can range from about 0.01-20 (w/w %), from about 0.1-15 (w/w %), and from about 1-11 (w/w %).

### Surfactants and Dispersants

To achieve a homogeneous dispersion of the substantially solid components of the formulation, surfactants and emulsifying agents such as saturated and unsaturated polyoxyethylene (2-80) alkyl (C8-20) ethers, for example Polysorbate 20-80, or polyethylene glycol ethers derived from castor oil, such as Ricinoleth-40 can be added. Other suitable surfactants include ethoxylated ethers of fatty alcohols (C16-C18), having 3-20 ethylene oxide units.

### Preservatives

Other suitable components include preservatives with antifungal and antimicrobial activity, including, but not limited to propionic acid, hexa-2,4-dienoic acid, benzoic acid, 2-hydroxy-benzoic acid, and their salts, esters of p-hydroxybenzoic acid, aromatic alcohols such as benzyl alcohol or phenoxyethanol, unsaturated and/or saturated fatty acids, such as undecylenic acid, lauric acid, oleic acid and/or stearic acid, sodium dehydroacetate, and other structural or functional equivalents, to prevent microbial growth and spoilage of the formulation during storage. The overall composition range for the specified surfactants and preservatives can typically range from about 0-10 (w/w %), from about 0.1-5 (w/w %), and from about 0.5-3 (w/w %).

### Other Performance Enhancing Agents

In addition to the aforementioned properties relevant to the functional performance of the formulation, complementary substances can be added to the mascara formulations to provide beneficial health effects that include among others, anti-microbial, anti-oxidant, anti-inflammatory, anti-aging, skin-conditioning, wound healing, and UV-blocking properties. Bioactive agents, nutrients and/or pharmaceutical compounds that promote the smoothing and/or moisturizing of the eyelashes and/or eyelid skin, that enhance blood perfusion and/or aid in hair growth are also contemplated for incorporation into the mascara formulations to provide a number of other performance enhancing effects desirable by a user. Such complementary substances can particularly include those of natural origin, so as to replace agents of purely synthetic nature, thus providing for a substantially more “organic” and healthier product character for avoiding hazardous chemicals typically involved in product formulation.

Naturally occurring resins, fragrances, oils, and waxes of plant or animal derived materials among others can be added to further enhance the performance of the mascara formulations. Suitable waxes for use in such embodiments can comprise of bee wax, lanolin, candelilla, soy, or carnauba wax. Fragrant and/or flavoring oils, obtained from apple, cherry, green tea, cinnamon, clove, black tea, plum, mango, date, watermelon, coconut, pear, jasmine, peach, fennel, fragrant melon, lychee, mint, chocolate, coffee, cream, banana, almond, grape, strawberry, blueberry, blackberry, pine, kiwi, sapote, taro, lotus, pineapple, orange, lemon, melon, peach, licorice, vanilla, rose, osmanthus, kiwi, ginseng, spearmint, citrus, cucumber, honeydew, walnut, almond, honey can be provided to provide to enhance the olfactory appeal to a user.

As one embodiment, anti-inflammatory agents can be added to the mascara formulations to prevent and treat skin inflammation. Suitable anti-inflammatory agents for incorporation into the mascara formulations can include NSAIDS and COX-2 inhibitors, such as curcumin extract, evodia extract, Camellia sinesis extract, cocoa seed butter, citric acid, salicylic acid, tocopherol, loganin, palmatine, Vitis vinifera extract, among other anti-inflammatory ingredients.

As another embodiment, anti-oxidants can be provided to the mascara formulation to prevent or reduce oxidative stress and/or collagen degradation occurring in the skin. Suitable anti-oxidants for incorporation into the mascara and eyeliner formulations can include Vitis vinifera extract, tocopherol and tocopherol derivatives, certain lycopenes such as β-carotene, certain anthocyans (blueberry extract, basil grape extract), certain catechins (green tea extract, grape seed extract, litchi extract), certain quercetins (blueberry extract, grape seed extract, apple extract), certain genistein extract (gingoko biloba extract, soja extract), acetyl hexapeptide-3, Silybum marianum fruit extract, or silymarin (milk thistle).

As one embodiment, anti-microbial ingredients obtained from natural and/or synthetic sources can be added to the mascara formulation to protect against the growth of microorganisms during storage, as well as regulate the natural microbial balance on the eyelid skin and eyelash hair. Suitable anti-microbial agents for incorporation into the magnetic mascara formulations can include imidazolidinyl urea, or certain extracts having anti-microbial activities such as Matricaria chamomilla extract, Aloe vera extract, Calendula officinalis extract, Kigelia africana extract, Lavandulla officinallis essential oil, Melaleuca alternifolia essential oil, or Cinnamomum zeylanicum essential oil.

Further, as several embodiments, the mascara formulations can comprise multiple skin and hair conditioning agents that improve moisture retention, and provide an appearance of suppleness to skin and additional volume to the hair. Suitable skin and hair conditioning agents for incorporation into the mascara formulations can include shea butter, pentylene glycol, acetyl hexapeptide-3, lipobelle soyaglycone, xylitylglucoside, anhydroxyxylitol, xylitol, ethylhexylglycerin, squalene, Triticum vulgare germ oil unsaponifiables, ceramide-3, ethylhexyl stearate, biosaccharide gum-1, paraffinum liquidum, isopropyl palminate, glycerin stearate, D-panthenol, lactic acid, tocopherol and tocopherol derivatives, cetearyl alcohol, dicaprylyl carbonate, butylene alcohol, butylene glycol, and carnosine, butylene glycol cocoate, propylheptyl caprylate, glycine soja sterols (soybean extracts), decyl oleate, PPG-26-buteth-26, potassium ascorbyl tocopheryl phosphate, guar hydroxypropyltrimonium chloride, methyl gluceth-10, avocado oil, hydrolyzed keratin or allantoin.

As another embodiment, moisture maintaining agents and agents that play a preventive role in skin-aging can be added. Suitable agents for incorporation into the mascara formulations can include polyunsaturated fatty acids, for example omega-3 fatty acids such as α-linolenic acid (ALA), eicosapentaenoic acid (EPA), and/or docosahexaenoic acid (DHA), as well as essential fatty acids (EFAs) or essential oils derived from plant sources, such as hemp oil.

In addition, as one embodiment, UV blocking agents can be added to prevent or reduce cellular damage that occurs upon UV exposure through the generation of reactive oxygen species that can accelerate skin aging. Suitable UV blocking agents for incorporation into the mascara formulations can include butylene glycol dicaprylyate or dicaprate, ethylhexyl methoxycinnamate, bis-ethylhexylloxyphenol methoxyphenyl triazine, camphor benzalkonium methosulfate, diethylhexyl butamido triazone, bisdisulizole disodium, drometrizole trisiloxane, ethylhexyl triazone, methyl anthranilate, 4-methylbenzylidene camphor, methylene bis-benzotriazolyl tetramethylbutyl phenol, octocrylene, butyl methoxydibenzoylmethane, phenylbenzimidazole sulfonic acid, polyacrylamidomethyl benzylidene camphor, or terephthalylidene dicamphor sulfonic acid.

Other suitable components include preservatives with antifungal and antimicrobial activity, including, but not limited to benzoic acid, propionic acid, and their salts, esters of p-hydroxybenzoic acid, aromatic alcohols such as benzyl alcohol or phenoxyethanol, unsaturated and/or saturated fatty acids, such as undecylenic acid, lauric acid, oleic acid and/or stearic acid, sodium dehydroacetate, and other structural or functional equivalents, to prevent microbial growth and spoilage of the formulation during storage. The overall composition range for the specified surfactants and preservatives can typically range from about 0-10 (w/w %), more preferably 0.1-5 (w/w %), and most preferably from about 0.5-3 (w/w %).

Several exemplary formulations for making the load-bearing magnetic mascara are further described in Examples 1-5. Alternative substitutions that are conceivable by persons skilled in the art of cosmetic formulations are contemplated within the scope of the present invention.

### D. Exemplary Formulations/Processes for Producing Magnetically Attachable Eyelash Prosthetics

The magnetically attachable eyelash prosthetic comprises: (a) a lash base incorporating one or more magnetic elements exhibiting high intrinsic coercivity; and (b) a plurality of eyelash hairs adhered at the proximal end to the lash base, wherein the magnetic elements are positioned towards the surface of the lash base capable of contacting and attaching to the surface of a magnetic film formed by pre-coating native lashes with a magnetic mascara formulation described herein and/or a magnetic eyeliner film formed over an eyelid. The “magnetic lash base,” “magnetic elements,” and “eyelash hairs” are further described below.

The “magnetic lash base” can be manufactured from a wide range of polymeric materials, provided that these materials can be formed to be substantially flexible and conforming to the skin, have adequate dermal compatibility without significant irritation potential, and are easily blended with the contemplated magnetic elements. Suitable polymers capable of forming an eyelash base include fluoroelastomers, polysulfones, polyamides, polyurethanes, polyesters, polyethers, silicones, polycarbonates, polyurethane carbonates, polyesters, polyamides, polyimides, polyvinyls, and polyolefins, polyvinyl alcohols, polyacetates, including blends and mixtures thereof.

Particularly suited for a skin-contacting application can be polyurethane and silicone-based materials, due to their intrinsically low irritation potential, good dermal compatibility and a widely adjustable range of flexibility. These polymer materials are available with a wide range of elastic properties, for example, that can be expressed through a measurement of shore hardness A, which can range from 0 to about 90 durometers. For the given application, ranges smaller than 50 durometers, more preferably smaller than 40 durometers and most preferably smaller than 30 durometers can be considered an optimum range of softness. In addition, the aforementioned polymers possess excellent compounding properties, and can be processed through a wide range of methods ideally suitable for blending with contemplated “magnetic elements.”

The magnetic elements are not limited as to the exact disposition of the polymer substrate utilized for forming the contemplated “magnetic lash base.” For example, the magnetic elements can be incorporated, adhered, layered, reacted, blended/mixed, embedded, grafted, bonded, compounded, crosslinked, copolymerized and/or reacted with the monomers for forming the polymer substrate, or can be processed as part of an intermediate layer that can be adhered, adjoined, affixed and/or reacted, or combined with the polymer substrate utilized for forming the “magnetic lash base” in any manner. Furthermore, the magnetic elements can be combined with a conventional polymer, and the combination can be adhered onto/around/ within the “magnetic lash base” or the surface of the device so that the magnetic element and the polymer substrate can be deposited simultaneously or sequentially. The magnetic elements can be incorporated into the polymer substrate of the “magnetic lash base” through various processes, including dispersion, suspension, sedimentation, encapsulation, coating, layering, film deposition, sputtering, spraying and similar functional equivalent. Depending on the particular process applied, the spatial distribution of the magnetic elements dispersed in the “magnetic lash base” can be substantially isotropic, or anisotropic, homogeneous, or inhomogeneous, or in the form of a gradient or non-gradient distribution.

Because the magnetic adherent force between the surface of a first ferromagnetic material (e.g., magnetic element) and the surface of a second ferromagnetic material (e.g., magnetic particles) (towards each other) depends on the orientation and distance between them, the smaller the gap existing between the “magnetic elements” incorporated into the “magnetic lash base” and the “magnetic particles” in the “magnetic mascara,” the stronger the resulting adherent force. Thus, the magnetic elements in the “magnetic lash base” are preferably arranged in close local proximity to the surface of the applied magnetic mascara and/or magnetic eyeliner. To achieve this specific spatial arrangement, the magnetic particles can be sedimented by gravitation within the monomer solution during the curing process of the polymer substrate utilized for forming the “magnetic lash base.” Alternatively, a magnetic force can be utilized to apply reverse gravitational pull on the magnetic elements when suspended in the monomer solution, such that the particles are preferably oriented not at the bottom, but at the top of the substrate.

The foregoing description, for purposes of explanation, refers to specific nomenclature to provide a thorough understanding of the invention. However, it will be apparent to one skilled in the art that the specific details are not required in order to practice the invention. The foregoing descriptions of specific embodiments of the present invention are presented for purpose of illustration and description. They are not intended to be exhaustive or to limit the invention to the precise forms disclosed. Obviously, many modifications and variations are possible in view of the above teachings. The embodiments are shown and described in order to best explain the principles of the invention and practical applications, to thereby enable others skilled in the art to best utilize the invention and various embodiments with various modifications as suitable for the particular uses contemplated. It is intended that the scope of the invention be defined by the following claims and their equivalents.

### EXAMPLES

### Example 1

### Exemplary Structural Adhesive Formulation for the Load-Bearing Magnetic Mascara Employing Iron (11,111) Oxide Nanocrystals (SPIO)

As several embodiments, exemplary structural adhesive formulations capable of stably adhering to native eyelashes can include the following agents:

| | | Composition: | | |
|---|---|---|---|---|
| | | A | B | C |
| Function | Ingredient(s) | (w/w) % | (w/w) % | (w/w) % |
| Magnetic Element(s) | Iron (II, III) oxide nanocrystals (SPIO) | 30.00 | 35.00 | 40.00 |
| Solvent(s) | Water | 30.00 | 30.00 | 30.00 |
| Adhesive agent(s) | Methacrylic acid-2-ethylhexyl acrylate | 20.00 | 15.00 | 10.00 |
| | copolymer | | | |
| | 2-Propenoic acid, Homopolymer | 1.000 | 0.500 | 0.250 |
| Viscosifying agent(s) | Butyl acrylate/methyl | 1.50 | 1.00 | 0.50 |
| | methacrylate/methacrylic acid | | | |
| | copolymer | | | |
| Gelling agent(s) | Hydroxyethylcellulose | 0.05 | 0.10 | 0.15 |
| Surfactant(s) | Alcohols, C16-18, ethoxylated (3->20 | ≤10.50 | ≤10.50 | ≤10.50 |
| | EO); Fatty acids, C16-18; Paraffin and | | | |
| | Hydrocarbon waxes | | | |
| Emollient/Humectant(s) | Alcohols, C14-18; Alcohols, C16-18; | ≤6.00 | ≤6.00 | ≤6.00 |
| | Propane-1,2,3-triol | | | |
| Preservative(s) | Benzyl alcohol, 2-Phenoxyethanol, | ≤1.00 | ≤1.00 | ≤1.00 |
| | Hexa-2,4-dienoic acid, | | | |
| | 2-Hydroxy-benzoic acid | | | |
| Pigment(s) | Titanium dioxide; Iron Oxides Yellow (CI | Balance | Balance | Balance |
| | 77492); Iron Oxides Red (CI 77491); | | | |
| | Iron Oxides Black (CI 77499) | | | |

### EXAMPLE 2

### Exemplary Structural Adhesive Formulation for the Load-Bearing Magnetic Mascara Employing Iron (II,III) Oxide Nanopowder (<100 nm Particle Size)

As several embodiments, exemplary structural adhesive formulations capable of stably adhering to native eyelashes can include the following agents:

| | | Composition: | | |
|---|---|---|---|---|
| | | D | E | F |
| Function | Ingredient(s) | (w/w) % | (w/w) % | (w/w) % |
| Magnetic Element(s) | Iron (II, III) oxide nanopowder, >50- | 30.00 | 35.00 | 40.00 |
| | 100 nm particle size | | | |
| Solvent(s) | Water | 30.00 | 30.00 | 30.00 |
| Adhesive agent(s) | Methacrylic acid-2-ethylhexyl acrylate | 20.00 | 15.00 | 10.00 |
| | copolymer | | | |
| | 2-Propenoic acid, Homopolymer | 1.000 | 0.500 | 0.250 |
| Viscosifying agent(s) | Butyl acrylate/methyl | 1.50 | 1.00 | 0.50 |
| | methacrylate/methacrylic acid | | | |
| | copolymer | | | |
| Gelling agent(s) | Hydroxyethylcellulose | 0.05 | 0.10 | 0.15 |
| Surfactant(s) | Alcohols, C16-18, ethoxylated (3->20 | ≤10.50 | ≤10.50 | ≤10.50 |
| | EO); Fatty acids, C16-18; Paraffin and | | | |
| | Hydrocarbon waxes | | | |
| Emollient/Humectant(s) | Alcohols, C14-18; Alcohols, C16-18; | ≤6.00 | ≤6.00 | ≤6.00 |
| | Propane-1,2,3-triol | | | |
| Preservative(s) | Benzyl alcohol, 2-Phenoxyethanol, | ≤1.00 | ≤1.00 | ≤1.00 |
| | Hexa-2,4-dienoic acid, | | | |
| | 2-Hydroxy-benzoic acid | | | |
| Pigment(s) | Titanium dioxide; Iron Oxides Yellow (CI | Balance | Balance | Balance |
| | 77492); Iron Oxides Red (CI 77491); | | | |
| | Iron Oxides Black (CI 77499) | | | |

### EXAMPLE 3

### Exemplary Structural Adhesive Formulation for the Load-Bearing Magnetic Mascara Employing Iron (II,III) Oxide Micronized Powder (<5 μm Particle Size)

As several embodiments, exemplary structural adhesive formulations capable of stably adhering to native eyelashes can include the following agents:

| | | Composition: | | |
|---|---|---|---|---|
| | | G | H | I |
| Function | Ingredient(s) | (w/w) % | (w/w) % | (w/w) % |
| Magnetic Element(s) | Iron (II, III) oxide micronized powder, <5 | 30.00 | 35.00 | 40.00 |
| | μm particle size | | | |
| Solvent(s) | Water | 30.00 | 30.00 | 30.00 |
| Adhesive agent(s) | Methacrylic acid-2-ethylhexyl acrylate | 20.00 | 15.00 | 10.00 |
| | copolymer | | | |
| | 2-Propenoic acid, Homopolymer | 1.000 | 0.500 | 0.250 |
| Viscosifying agent(s) | Butyl acrylate/methyl | 1.50 | 1.00 | 0.50 |
| | methacrylate/methacrylic acid | | | |
| | copolymer | | | |
| Gelling agent(s) | Hydroxyethylcellulose | 0.05 | 0.10 | 0.15 |
| Surfactant(s) | Alcohols, C16-18, ethoxylated (3->20 | ≤10.50 | ≤10.50 | ≤10.50 |
| | EO); Fatty acids, C16-18; Paraffin and | | | |
| | Hydrocarbon waxes | | | |
| Emollient/Humectant(s) | Alcohols, C14-18; Alcohols, C16-18; | ≤6.00 | ≤6.00 | ≤6.00 |
| | Propane-1,2,3-triol | | | |
| Preservative(s) | Benzyl alcohol, 2-Phenoxyethanol, | ≤1.00 | ≤1.00 | ≤1.00 |
| | Hexa-2,4-dienoic acid, | | | |
| | 2-Hydroxy-benzoic acid | | | |
| Pigment(s) | Titanium dioxide; Iron Oxides Yellow (CI | Balance | Balance | Balance |
| | 77492); Iron Oxides Red (CI 77491); | | | |
| | Iron Oxides Black (CI 77499) | | | |

### EXAMPLE 4

### Exemplary Structural Adhesive Formulation for the Load-Bearing Magnetic Mascara Employing Neodymium-Iron-Boron Nd₂Fe₁₄B Powder (>50 μm Particle Size)

As several embodiments, exemplary structural adhesive formulations capable of stably adhering to native eyelashes can include the following agents:

| | | Composition: | | |
|---|---|---|---|---|
| | | J | K | L |
| Function | Ingredient(s) | (w/w) % | (w/w) % | (w/w) % |
| Magnetic Element(s) | Neodymium-iron-boron Nd2Fe14B | 30.00 | 35.00 | 40.00 |
| | powder, >50-60 μm particle size | | | |
| Solvent(s) | Water | 30.00 | 30.00 | 30.00 |
| Adhesive agent(s) | Methacrylic acid-2-ethylhexyl acrylate | 20.00 | 15.00 | 10.00 |
| | copolymer | | | |
| | 2-Propenoic acid, Homopolymer | 1.000 | 0.500 | 0.250 |
| Viscosifying agent(s) | Butyl acrylate/methyl | 1.50 | 1.00 | 0.50 |
| | methacrylate/methacrylic acid | | | |
| | copolymer | | | |
| Gelling agent(s) | Hydroxyethylcellulose | 0.05 | 0.10 | 0.15 |
| Surfactant(s) | Alcohols, C16-18, ethoxylated (3->20 | ≤10.50 | ≤10.50 | ≤10.50 |
| | EO); Fatty acids, C16-18; Paraffin and | | | |
| | Hydrocarbon waxes | | | |
| Emollient/Humectant(s) | Alcohols, C14-18; Alcohols, C16-18; | ≤6.00 | ≤6.00 | ≤6.00 |
| | Propane-1,2,3-triol | | | |
| Preservative(s) | Benzyl alcohol, 2-Phenoxyethanol, | ≤1.00 | ≤1.00 | ≤1.00 |
| | Hexa-2,4-dienoic acid, | | | |
| | 2-Hydroxy-benzoic acid | | | |
| Pigment(s) | Titanium dioxide; Iron Oxides Yellow (CI | Balance | Balance | Balance |
| | 77492); Iron Oxides Red (CI 77491); | | | |
| | Iron Oxides Black (CI 77499) | | | |

### EXAMPLE 5

### Exemplary Structural Adhesive Formulation for the Load-Bearing Magnetic Mascara Employing Barium Ferrite BaFe₁₂O₁₉ Powder (>50 μm Particle Size)

As several embodiments, exemplary structural adhesive formulations capable of stably adhering to native eyelashes can include the following agents:

| | | Composition: | | |
|---|---|---|---|---|
| | | M | N | O |
| Function | Ingredient(s) | (w/w) % | (w/w) % | (w/w) % |
| Magnetic Element(s) | Barium ferrite BaFe12O19 powder, >40- | 30.00 | 35.00 | 40.00 |
| | 50 μm particle size | | | |
| Solvent(s) | Water | 30.00 | 30.00 | 30.00 |
| Adhesive agent(s) | Methacrylic acid-2-ethylhexyl acrylate | 20.00 | 15.00 | 10.00 |
| | copolymer | | | |
| | 2-Propenoic acid, Homopolymer | 1.000 | 0.500 | 0.250 |
| Viscosifying agent(s) | Butyl acrylate/methyl | 1.50 | 1.00 | 0.50 |
| | methacrylate/methacrylic acid | | | |
| | copolymer | | | |
| Gelling agent(s) | Hydroxyethylcellulose | 0.05 | 0.10 | 0.15 |
| Surfactant(s) | Alcohols, C16-18, ethoxylated (3->20 | ≤10.50 | ≤10.50 | ≤10.50 |
| | EO); Fatty acids, C16-18; Paraffin and | | | |
| | Hydrocarbon waxes | | | |
| Emollient/Humectant(s) | Alcohols, C14-18; Alcohols, C16-18; | ≤6.00 | ≤6.00 | ≤6.00 |
| | Propane-1,2,3-triol | | | |
| Preservative(s) | Benzyl alcohol, 2-Phenoxyethanol, | ≤1.00 | ≤1.00 | ≤1.00 |
| | Hexa-2,4-dienoic acid, | | | |
| | 2-Hydroxy-benzoic acid | | | |
| Pigment(s) | Titanium dioxide; Iron Oxides Yellow (CI | Balance | Balance | Balance |
| | 77492); Iron Oxides Red (CI 77491); | | | |
| | Iron Oxides Black (CI 77499) | | | |

We claim:

1. A lash-adhering, load-bearing magnetic mascara comprising:
a cosmetic base that includes, at about 10 to 60 percent by weight, magnetic particles incorporating a ferromagnetic compound comprising one or more transition metal oxides, post-transition metal oxides, alkaline earth metal oxides, or combinations thereof, having intrinsic coercivities (Hci) ranging from about 10 kA/m to 25,000 kA/m; and
a lash-adhering, load-bearing structural adhesive selected from polyurethane polymers and blends, acrylic polymers and blends, silicone polymers and blends, and vinyl polymers and blends, and
that converts from a gel state to a load-bearing adhesive state after application to a lash surface, forming a lash-adhered, magnetic-mascara film that includes
an interior adhesive surface contoured over the lash surface and
an exterior non-adhesive surface for reversibly and magnetically attaching one or more magnetic eyelash prosthetics.

2. The lash-adhering, load-bearing magnetic mascara of claim 1 further comprising a pigment selected from the group consisting of natural pigments, synthetic pigments, metallic pigments, non-metallic pigments, inorganic pigments, organic pigments, magnetic pigments, and pearlescent pigments.

3. The lash-adhering, load-bearing magnetic mascara of claim 2, wherein the pigment is inorganic, and selected from the group consisting of black iron oxides, yellow iron oxides, red iron oxides, brown iron oxides, titanium dioxide, manganese violet, ultramarine blue, chromium oxide, and ferric blue.

4. The lash-adhering, load-bearing magnetic mascara of claim 2, wherein the pigment is a particle having an average diameter size selected from the group consisting of ranges from about 1 μm to 1 mm, from about 1 μm to 500 μm, from about 500 μm to 1 mm, and from about 1 μm to 100 μm.

5. (canceled)

6. The lash-adhering, load-bearing magnetic mascara of claim 1, wherein the ferromagnetic compound is selected from the group consisting of iron oxides, Hematite Fe₂O₃, Magnetite-Fe^{II}(Fe^{III})₂O₄, Super-paramagnetic iron oxides, Chromium dioxide CrO₂, Barium ferrites, BaFe₁₂O₁₉, Ba₂ZnFe₁₈O₂₃, BaFe²⁺₂Fe³⁺₁₆O₂₇, Bismuth ferrite (BiFeO₃), Manganese-zinc ferrite, MnZn, Cobalt ferrite, CoFe₂O₄, Strontium ferrite, SrFe₁₂O₁₉, and Yttrium iron granate Y₃Fe₅O₁₂.

7. The lash-adhering, load-bearing magnetic mascara of claim 6, wherein the iron oxide is black iron oxide with a concentration from about 10-60 (w/w %), from about 20-50 (w/w %), or from about 25-45 (w/w %).

8. The lash-adhering, load-bearing magnetic mascara of claim 1, wherein the magnetic particles exhibit an average diameter size selected from the group consisting of ranges from about 1 μm to 1 mm, from about 1 μm to 500 μm, from about 500 μm to 1 mm, and from about 1 μm to 100 μm.

9. The lash-adhering, load-bearing magnetic mascara of claim 1, wherein the magnetic particles are encapsulated and exhibit an average diameter size ranging from about 100 nm to 1 μm.

10. (canceled)

11. The lash-adhering, load-bearing magnetic mascara of claim 1, wherein the curable, structural adhesive comprises a fluorinated acrylate polymer.

12. (canceled)

13. The lash-adhering load-bearing magnetic mascara of claim 1 further comprising a fiber selected from the group consisting of natural fibers, synthetic fibers, and combinations thereof.

14. The lash-adhering, load-bearing magnetic mascara of claim 1 further comprising at least one ingredient selected from the group consisting of lash growth-promoting serums, conditioners, anti-oxidants, and essential oils.

15. (canceled)

16. (canceled)

17. (canceled)

18. A magnetically attachable eyelash prosthetic system mediated by a magnetic mascara, the prosthetic system comprising:
a lash-adhering, load-bearing magnetic mascara comprising a cosmetic base that includes, at about 10 to 60 percent by weight, magnetic particles incorporating a ferromagnetic compound comprising one or more transition metal oxides, post-transition metal oxides, alkaline earth metal oxides, or combinations thereof, having intrinsic coercivities (Hci) ranging from about 10 kA/m to 25,000 kA/m, and a lash-adhering, load-bearing structural adhesive selected from polyurethane polymers and blends, acrylicpolymers and blends, silicone polymers and blends, and vinyl polymers and blends; and
a magnetic eyelash prosthetic incorporating one or more magnetic elements comprising transition elements, post-transition elements, lanthanide elements or combinations thereof, having intrinsic coercivities (Hci) ranging from about 10 kA/m to 25,000 kA/m and positioned at least at the lash base of the eyelash prosthetic.

19. The magnetically attachable eyelash prosthetic system of claim 18, wherein the magnetic mascara further comprises a pigment selected from the group consisting of natural pigments, synthetic pigments, metallic pigments, non-metallic pigments, inorganic pigments, organic pigments, magnetic pigments, and pearlescent pigments.

20. (canceled)

21. The magnetically attachable eyelash prosthetic system of claim 18, wherein the ferromagnetic compound is selected from the group consisting of iron oxides, Hematite Fe₂O₃, Magnetite-Fe^{II}(Fe^{III})₂O₄, Super-paramagnetic iron oxides, Chromium dioxide CrO₂, Barium ferrites, BaFe₁₂O₁₉, Ba₂ZnFe₁₈O₂₃, BaFe²⁺₂Fe³⁺₁₆O₂₇, Bismuth ferrite (BiFeO₃), Manganese-zinc ferrite, MnZn, Cobalt ferrite, CoFe₂O₄, Strontium ferrite, SrFe₁₂O₁₉, and Yttrium iron granate

22. (canceled)

23. (canceled)

24. The magnetically attachable eyelash prosthetic system of claim 18, wherein the magnetic element is formed from one or more hard ferromagnetic materials comprising one or more rare-earth compounds.

25. The magnetically attachable eyelash prosthetic system of claim 18, wherein the ferromagnetic compound is selected from the group consisting of bismanol and AlNiCo.

26. The magnetically attachable eyelash prosthetic system of claim 24, wherein the rare-earth compound is neodymium-iron-boron Nd₂Fe₁₄B.

27. The magnetically attachable eyelash prosthetic system of claim 18, wherein the lash-adhering, load-bearing structural adhesive comprises a fluorinated acrylate polymer.

28. (canceled)

29. A method for making a magnetically attachable eyelash prosthetic system mediated by a magnetic mascara, the method comprising:
combining together a cosmetic base, at about 10 to 60 percent per weight, with magnetic particles incorporating ferromagnetic compounds having intrinsic coercivities (Hci) ranging from about 10kA/m to 25,000 kA/m and a lash-adhering, load-bearing structural adhesive selected from polyurethane polymers and blends, acrylic polymers and blends, silicone polymers and blends, and vinyl polymers and blends to produce a lash-adhering, load-bearing magnetic mascara; and
positioning one or more magnetic elements, having intrinsic coercivities (Hci) ranging from about 10 kA/m to 25,000 kA/m, at a lash base of the eyelash prosthetic to produce a magnetic eyelash prosthetic.

30. (canceled)
